(19) United States
(12) Patent Application Publication
(10) Pub. No.: US 2018/0346394 A1
(43) Pub. Date: Dec. 6, 2018

(54) ALKANE AROMATIZATION BY OXIDATIVE DEHYDROGENATION WITH CO2

(71) Applicant: SABIC Global Technologies B.V., Bergen op Zoom (NL)

(72) Inventors: Khalid A. Almusaiteer, Thuwal (SA); Abdulrahman Al-Hazmi, Thuwal (SA); Abdulaziz Al-Amer, Thuwal (SA); Ugo Ravon, Thuwal (SA)

(21) Appl. No.: 15/778,833

(22) PCT Filed: Nov. 28, 2016
(86) PCT No.: PCT/IB2016/057167

§ 371 Date: May 24, 2018

Related U.S. Application Data
(60) Provisional application No. 62/263,091, filed on Dec. 4, 2015.

(51) Int. Cl. C07C 2/76; B01J 29/48; B01J 29/40; B01J 29/46

(52) U.S. Cl. CPC C07C 2/76; B01J 29/48; B01J 29/405; C07C 15/08; B01J 2219/00166; C07C 2529/40; B01J 29/46

(57) ABSTRACT

An oxidative aromatization method and systems capable of producing aromatic hydrocarbons from an alkane or mixtures of alkanes and carbon dioxide (CO₂) are disclosed. Specifically, the method and systems use zeolite catalysts to catalyze the aromatization reaction of butane in the presence of CO₂. The aromatization reactions provide product streams with high single pass aromatic selectivity of benzene, toluene and xylenes (BTX).

### CROSS REFERENCE TO RELATED APPLICATIONS

This application claims benefit to U.S. Provisional Application No. 62/263,091 titled “Alkane Aromatization by Oxidative Dehydrogenation with CO₂” filed Dec. 4, 2015, which is incorporated herein in its entirety.

### BACKGROUND OF THE INVENTION

### A. Field of the Invention

The invention generally concerns an oxidative aromatization method and systems capable of catalyzing an alkane or a mixture of alkanes and carbon dioxide (CO₂) under reaction conditions sufficient to produce an aromatic hydrocarbon containing product stream. In particular, heterogeneous zeolite catalyst compositions are used to catalyze the production of benzene, toluene, and xylenes (BTX) from butane and CO₂. The disclosed oxidative aromatization method and systems provide product streams with high single pass aromatic hydrocarbon selectivity.

### B. Description of Related Art

Aromatic hydrocarbons are essential basic building blocks for a large number of petrochemical processes. The most important aromatic hydrocarbons are also the most simple, including benzene, toluene, and xylenes (BTX). These aromatic hydrocarbons are typically produced by catalytic reforming, coal tar processing, toluene disproportionation (TDP), and transalkylation (TA). The composition and yield of the final BTX product depends largely on the source feedstock. In recent years, the global aromatic hydrocarbons demand has far exceeded supply and demands are projected to increase for the constituents of BTX. Naphtha, which is used in catalytic reforming, is being substituted with shale gas (e.g. methane) as a cheaper feedstock, thereby resulting in a decreased volume of BTX produced from steam reforming. Aromatization of light hydrocarbons, in particular liquefied petroleum gas (LPG), which contains primarily methane, has grown in popularity in academic and industrial settings. Current processes for the conversion of LPG into BTX include M-2 Forming (Mobil), Cyclar (BP-UOP), Aroforming (IFB Salutec), Z-Forming Process (Mitsubishi), and Alpha process (Sanyo/Asahi). Most of these investigations have focused on the use of ZSM-5 based catalyst, which are strongly acidity and have good stability. By way of example, U.S. Patent Application Publication Nos. 2011/0257452 and 2011/0253596 to Khabashesku et al. disclose the use of zeolite catalysts for the conversion of lower alkanes to aromatic hydrocarbons. In another example, U.S. Patent Application Publication Nos. 2015/0099914 to Garza et al. and 2008/0293980 to Kiesslich et al. describe the use of H-ZSM-5 catalysts for the conversion of natural gas to aromatic hydrocarbons. In still another example, Shpiro et al., (Applied Catalysis A: General, 1994, 107:165-180) and Zavoianu et al. (Prog. Catal., 2003, Vol. 12, No. 1, pp. 69-82.) describe the conversion of butane to aromatic hydrocarbons using a zeolite catalyst.

Despite all the currently available efforts produce BTX compounds from alkanes, many of the current processes suffer from poor selectivity, increased formation of by-products, and decreased BTX yields or combinations thereof.

### SUMMARY OF THE INVENTION

A discovery has been made that solves the problems associated with the production of benzene, toluene, and xylenes (BTX) from an alkane or mixtures of alkanes in the presence of carbon dioxide (CO₂) and a zeolite catalyst. It has been surprisingly found that adding CO₂ to the reactant feed enhances aromatic hydrocarbon yield by lowering the formation of undesired side products and enhancing the formation of olefinic intermediates, which further enhances the yields of aromatic hydrocarbon compounds such as those constituting BTX. Without wishing to be bound by theory it is believed that CO₂ acts as a soft oxidant and hydrogen scavenger to inhibit side reactions and remove coking from the catalyst surface, which further improves the yield of benzene and other aromatic hydrocarbons. The CO₂ provides sufficient acidity to dehydrogenate the alkane to form olefinic intermediates. Furthermore, the oxidative aromatization method and systems of the present invention provide increased selectivity of benzene, toluene, and xylenes where the combined selectivity of benzene, toluene, and xylenes is at least 20% at a reaction temperature of 200° C. or at least 50% at a reaction temperature of 325° C. to 600° C., at 54 mole % of CO₂, and the conversion of butane is at least 15% at a reaction temperature of 200° C. or at least 80% at a reaction temperature of 325° C. to 450° C., at 10 mole % to 54 mole % of CO₂.

In one aspect of the present invention, there is disclosed an oxidative aromatization method for producing aromatic hydrocarbons from alkanes, the method including contacting a reactant feed that includes an alkane, or a mixture of alkanes, and CO₂ with a zeolite catalyst under reaction conditions sufficient to produce an aromatic hydrocarbon containing product stream. The amount CO₂ present in the reactant feed can be sufficient to dehydrogenate the alkane or mixtures of alkanes and the reactant feed can include at least 10 mole % of CO₂, preferably 10 mole % to 54 mole % of CO₂. The mole % ratio of CO₂ to the alkane or CO₂ to the mixture of alkanes is 0.1 to 10, preferably 0.1 to 7, more preferably 0.1 to 5, or most preferably 0.1 to 3. In another aspect of the method the reactant feed can include substantially stoichiometric amounts of CO₂. The alkane used in the method can be ethane, propane, butane or the mixture of alkanes includes at least 50, 60, 70, 80, or 90 mole %, or more, of the alkane. The butane can be n-butane, iso-butane or both. In another aspect of the method, the aromatic hydrocarbon containing product stream can include benzene, toluene, and xylenes (BTX), where the combined selectivity of benzene, toluene, and xylenes is at least 20% at a reaction temperature of 200° C. or at least 50% at a reaction temperature of 325° C. to 600° C., at 54 mole % of CO₂. The conversion of butane can be at least 15% at a reaction temperature of 200° C. or at least 80% at a reaction temperature of 325° C. to 375° C., at 10 mole % to 54 mole % of CO₂. In some aspects the reaction conditions include a temperature of at least 200° C., preferably 200° C. to 700° C., more preferably 200° C. to 600° C., even more preferably 250° C. to 550° C., or most preferably 275° C. to 500° C. The reaction conditions can further include a pressure of 0.5 to 5 bar, or 1 to 3 bar, and a gas hourly space velocity (GHSV) of 500 h⁻¹ to 10,000 h⁻¹, preferably 500 h⁻¹ to 5,000 h⁻¹. The reactants in the reactant feed can be in the gas phase and the reactant feed can further include a carrier gas. The carrier gas can be helium, argon, nitrogen, CO₂, or mixture thereof. In one embodiment the aromatic hydrocarbon containing product stream can include benzene, toluene, and xylenes. In another embodiment the reactant feed consists essentially of or consists of the alkane, or a mixture of alkanes, and carbon dioxide (CO₂) and the reactant feed does not include any other alkane other than butane. In a particular embodiment the reactant feed does not include oxygen (O₂). The zeolite catalyst employed in the method can be a regular zeolite or a hollow zeolite catalyst (e.g., MFI, FAU, ITH BEA, MOR, LTA, MWW, CHA, MRE, MFE, or a VFI zeolite catalyst), preferably an MFI zeolite catalyst, or a hollow MFI zeolite catalyst. The MFI zeolite catalyst can be ZSM-5, H-ZSM-5, a hollow ZSM-5, or a hollow H-ZSM-5 loaded with a catalytic metal or metal oxide thereof. The catalytic metal or metal oxide can include a Column 1, 2 and 6-14 (Groups IA, IIA, VIB, VIIB, VIII, VIIIB, IB, IIB, IIIA, IVA, or VA) metal or metal oxide of the Periodic Table, for example sodium (Na), magnesium (Mg), lantheum (La), Ytterbium (Y), vanadium (V), niobium (Nb), molybdenum (Mo), chromium (Cr), manganese (Mn), iron (Fe), cobalt (Co), nickel (Ni), copper (Cu), zinc (Zn), gallium (Ga), indium (In), tin (Sn), antimony (Sb), bismuth (Bi), tellurium (Te), and in a specific embodiment the metal is Fe. The zeolite catalyst can include up to 20 wt. % of the metal or metal oxide, preferably 0.1 wt. % to 20 wt. %, more preferably 1 wt. % to 10 wt. %, or most preferably 3 wt. % to 7 wt. % or about 5 wt. %. In another aspect, the zeolite catalyst has a Si/Al ratio of less than 100, preferably 5 to 75, more preferably 10 to 60, or most preferably 20 to 55. Without limiting the method to the production aromatic hydrocarbons alone, the method can further include collecting or storing the aromatic hydrocarbon containing product stream and using the produced aromatic hydrocarbon containing product stream to produce a petrochemical or a polymer.

Also disclosed is a system for producing aromatic hydrocarbons using the methods described herein, the system includes an inlet for a reactant feed that includes an alkane or a mixture of alkanes and carbon dioxide (CO₂); a reaction zone that is configured to be in fluid communication with the inlet; and an outlet configured to be in fluid communication with the reaction zone to remove an aromatic hydrocarbon containing product stream. The reaction zone can include the reactant feed and a zeolite catalyst, and an amount of CO₂ sufficient to dehydrogenate the alkane of mixture of alkanes. The system can further include a collection device that is capable of collecting the aromatic hydrocarbon containing product stream and the reaction zone can be a continuous flow reactor selected from a fixed-bed reactor, a fluidized reactor, or a moving bed reactor. The mole % ratio of CO₂ to the alkane or CO₂ to the mixture of alkanes is 0.1 to 10, preferably 0.1 to 7, more preferably 0.1 to 5, or most preferably 0.1 to 3, and the reactant feed can include at least 10 mole % of CO₂, preferably 10 mole % to 54 mole % of CO₂. In another aspect of the system the reactant feed can include substantially stoichiometric amounts of CO₂. The alkane used in the system can be butane or the mixture of alkanes includes at least 50, 60, 70, 80, or 90 mole %, or more, of butane. The butane can be n-butane. In some aspects of the system, the temperature of the reaction is at least 200° C., preferably 200° C. to 700° C., more preferably 200° C. to 600° C., even more preferably 250° C. to 550° C., or most preferably 275 to 540° C. The reaction conditions can further include a pressure of 0.5 to 5 bar, or 1 to 3 bar.

The reactants in the reactant feed of the system can be in the gas phase and the reactant feed can further include a carrier gas. The carrier gas can be helium, argon, nitrogen, CO₂, or mixtures thereof. In one embodiment the aromatic hydrocarbon containing product stream can include benzene, toluene, and xylenes. In another embodiment the reactant feed consists essentially of or consists of the alkane, or a mixture of alkanes, and carbon dioxide (CO₂) and the reactant feed does not include any other alkane other than butane. In a particular embodiment the reactant feed does not include oxygen (O₂). The zeolite catalyst employed in the system can be can be a regular zeolite or a hollow zeolite catalyst (e.g., MFI, FAU, ITH BEA, MOR, LTA, MWW, CHA, MRE, MFE, or a VFI zeolite catalyst), preferably an MFI zeolite catalyst, or a hollow MFI zeolite catalyst. The MFI zeolite catalyst can be ZSM-5, H-ZSM-5, a hollow ZSM-5, or a hollow H-ZSM-5 loaded with a catalytic metal or metal oxide. The catalytic metal or metal oxide can include a Column 1, 2 and 6-14 (Groups IA, IIA, VIB, VIIB, VIII, VIIIB, IB, IIB, IIIA, IVA, or VA) metal or metal oxide of the Periodic Table, for example Cr, Mn, Fe, Co, Ni, Cu, Zn, Ga, Sn, Sb, Te, and in a specific embodiment the metal is Fe. The zeolite catalyst can include up to 10 wt. % of the metal or metal oxide, preferably 0.1 wt. % to 20 wt. %, more preferably 1 wt. % to 10 wt. %, or most preferably 3 wt. % to 7 wt. % or about 5 wt. %. In another aspect of the system, the zeolite catalyst has a Si/Al ratio of less than 100, preferably 5 to 75, more preferably 10 to 60, or most preferably 20 to 55.

In the context of the present invention, 51 embodiments are described. Embodiment 1 is an oxidative aromatization method for producing aromatic hydrocarbons from alkanes, the method can include contacting a reactant feed comprising an alkane, or a mixture of alkanes, and carbon dioxide (CO₂) with a zeolite catalyst under reaction conditions sufficient to produce an aromatic hydrocarbon containing product stream, wherein the amount of CO₂ present in the reactant feed is sufficient to dehydrogenate the alkane or mixture of alkanes, and wherein the reactant feed comprises at least 10 mole % of CO₂, preferably 10 mole % to 54 mole % of CO₂. Embodiment 2 is the method of embodiment 1, wherein wherein the mole % ratio of CO₂ to the alkane or CO₂ to the mixture of alkanes is 0.1 to 10, preferably 0.1 to 7, more preferably 0.1 to 5, or most preferably 0.1 to 3. Embodiment 3 is the method of any one of embodiments 1 to 2, wherein the reactant feed comprises substantially stoichiometric amounts of CO₂. Embodiment 4 is the method of any one of embodiments 1 to 3, wherein the alkane is butane or wherein the mixture of alkanes comprises at least 50, 60, 70, 80, or 90 mole %, or more, of butane. Embodiment 5 is the method of embodiment 4, wherein the butane is n-butane. Embodiment 6 is the method of embodiment 5, wherein the aromatic hydrocarbon containing product stream comprises benzene, toluene, and xylene, and wherein the combined selectivity of benzene, toluene, and xylene is at least 20% at a reaction temperature of 200° C. or at least 50% at a reaction temperature of 325° C. to 600° C., at 54 mole % of CO₂. Embodiment 7 is the method of embodiment 6, wherein the conversion of butane is at least 15% at a reaction temperature of 200° C. or at least 80% at a reaction temperature of 325° C. to 450° C., at 10 mole % to 54 mole % of CO₂. Embodiment 8 is the method of any one of embodiments 1 to 7, wherein the reaction conditions include a temperature of at least 200° C., preferably 200° C. to 700° C., more preferably 200° C. to 600° C., even more preferably 250° C. to 550° C., or most preferably 275 to 540° C. Embodiment 9 is the method of embodiment 8, wherein the reaction conditions further include a pressure of 0.5 to 5 bar, or 1 to 3 bar, and a gas hourly space velocity (GHSV) of 500 h⁻¹ to 10,000 h⁻¹, preferably 500 h⁻¹ to 5,000 h⁻¹. Embodiment 10 is the method of any one of embodiments 1 to 9, wherein the reactants in the reactant feed are in the gas phase. Embodiment 11 is the method of embodiment 10, wherein the reactant feed further comprises a carrier gas. Embodiment 12 is the method of embodiment 11, wherein the carrier gas is helium, argon, nitrogen, CO₂, or mixtures thereof. Embodiment 13 is the method of any one of embodiments 1 to 12, wherein the aromatic hydrocarbon containing product stream comprises benzene, toluene, and xylene. Embodiment 14 is the method of any one of embodiments 1 to 13, wherein the reactant feed consists essentially of or consists of the alkane, or a mixture of alkanes, and carbon dioxide (CO₂). Embodiment 15 is the method of any one of embodiments 1 to 14, wherein the reactant feed does not include any other alkane other than butane. Embodiment 16 is the method of any one of embodiments 1 to 15, wherein the reactant feed does not include oxygen (O₂). Embodiment 17 is the method of any one of embodiments 1 to 16, wherein the zeolite catalyst is a MFI, FAU, ITH BEA, MOR, LTA, MWW, CHA, MRE, MFE, or a VFI zeolite catalyst, preferably a MFI zeolite catalyst, or a hollow MFI zeolite catalyst. Embodiment 18 is the method of embodiment 17, wherein the MFI zeolite catalyst is ZSM-5, H-ZSM-5, a hollow ZSM-5, or a hollow H-ZSM-5. Embodiment 19 is the method of any one of embodiments 1 to 18, wherein the zeolite catalyst is loaded with a catalytic metal or metal oxide. Embodiment 20 is the method of embodiment 19, wherein the catalytic metal or metal oxide is a group IA, IIA, VIB, VIIB, VIII, VIIIB, IB, IIB, IIIA, IVA, or VA metal or metal oxide. Embodiment 21 is the method of embodiment 20, wherein the metal or metal oxide comprises sodium (Na), magnesium (Mg), lantheum (La), Ytterbium (Y), vanadium (V), niobium (Nb), molybdenum (Mo), chromium (Cr), manganese (Mn), iron (Fe), cobalt (Co), nickel (Ni), copper (Cu), zinc (Zn), gallium (Ga), indium (In), tin (Sn), antimony (Sb), bismuth (Bi), tellurium (Te), or any combination thereof. Embodiment 22 is the method of embodiment 21, wherein the metal or metal oxide comprises Fe. Embodiment 23 is the method of any one of embodiments 19 to 22, wherein the zeolite catalyst comprises up to 20 wt. % of the metal or metal oxide, preferably 0.1 wt. % to 20 wt. %, more preferably 1 wt. % to 10 wt. %, or most preferably 3 wt. % to 7 wt. % or about 5 wt. %. Embodiment 24 is the method of any one of embodiments 1 to 23, wherein the zeolite catalyst has a Si/Al ratio of less than 100, preferably 5 to 75, more preferably 10 to 60, or most preferably 20 to 55. Embodiment 25 is the method of any one of embodiments 1 to 24, further comprising collecting or storing the aromatic hydrocarbon containing product stream. Embodiment 26 is the method of any one of embodiments 1 to 25, further comprising using the produced aromatic hydrocarbon containing product stream to produce a petrochemical or a polymer.

Embodiment 27 is a system for producing aromatic hydrocarbons, the system can include an inlet for a reactant feed comprising an alkane, or a mixture of alkanes, and carbon dioxide (CO₂), wherein the amount of CO₂ present in the reactant feed is sufficient to dehydrogenate the alkane or mixture of alkanes; a reaction zone that is configured to be in fluid communication with the inlet, wherein the reaction zone comprises the reactant feed and a zeolite catalyst; and an outlet configured to be in fluid communication with the reaction zone to remove an aromatic hydrocarbon containing product stream. Embodiment 28 is the system of embodiment 27, further comprising a collection device that is capable of collecting the aromatic hydrocarbon containing product stream. Embodiment 29 is the system of any one of embodiments 27 to 28, wherein the reaction zone is a continuous flow reactor selected from a fixed-bed reactor, a fluidized reactor, or a moving bed reactor. Embodiment 30 is the system of any one of claims 27 to 29, wherein the mole % ratio of CO₂ to the alkane or CO₂ to the mixture of alkanes is 0.1 to 10, preferably 0.1 to 7, more preferably 0.1 to 5, or most preferably 0.1 to 3. Embodiment 31 is the system of anyone of embodiments 27 to 30, wherein the reactant feed comprises at least 10 mole % of CO₂, preferably 10 mole % to 54 mole % of CO₂. Embodiment 32 is the system of any one of embodiments 27 to 31, wherein the reactant feed comprises substantially stoichiometric amounts of CO₂. Embodiment 33 is the system of any one of embodiments 27 to 32, wherein the alkane is ethane, propane, butane or any mixture thereof, and wherein the mixture of alkanes comprises at least 50, 60, 70, 80, or 90 mole %, or more, of ethane, propane, butane, or any combination thereof. Embodiment 32 is the system of embodiment 33, wherein the alkane is n-butane. Embodiment 33 is the system of any one of embodiments 27 to 34, wherein the temperature of the reaction zone is at least 200° C., preferably 200° C. to 700° C., more preferably 200° C. to 600° C., even more preferably 250° C. to 550° C., or most preferably 275 to 540° C. Embodiment 36 is the system of embodiment 35, wherein the pressure of the reaction zone is 0.5 to 5 bar, or 1 to 3 bar. Embodiment 37 is the system of any one of embodiments 27 to 36, wherein the reactants in the reactant feed are in the gas phase. Embodiment 38 is the system of embodiment 37, wherein the reactant feed further comprises a carrier gas. Embodiment 39 is the system of embodiment 38, wherein the carrier gas is helium, argon, nitrogen, CO₂, or mixtures thereof. Embodiment 40 is the system of any one of embodiments 27 to 39, wherein the aromatic hydrocarbon containing product stream comprises benzene, toluene, and xylene. Embodiment 41 is the system of any one of embodiments 27 to 40, wherein the reactant feed consists essentially of or consists of the alkane, or a mixture of alkanes, and carbon dioxide (CO₂). Embodiment 42 is the system of any one of embodiments 27 to 41, wherein the reactant feed does not include any other alkane other than butane. Embodiment 43 is the system of any one of embodiments 27 to 42, wherein the reactant feed does not include oxygen (O₂). Embodiment 44 is the system of any one of embodiments 27 to 43, wherein the zeolite catalyst is a MFI, FAU, ITH BEA, MOR, LTA, MWW, CHA, MRE, MFE, or a VFI zeolite catalyst, preferably a MFI zeolite catalyst, or a hollow MFI zeolite catalyst. Embodiment 45 is the system of embodiment 44, wherein the zeolite catalyst is ZSM-5 or H-ZSM-5, a hollow ZSM-5, or a hollow H-ZSM-5. Embodiment 46 is the system of any one of embodiments 27 to 45, wherein the zeolite catalyst is loaded with a catalytic metal or metal oxide. Embodiment 47 is the system of embodiment 46, wherein the catalytic metal or metal oxide is a group IA, IIA, VIB, VIIB, VIII, VIIIB, IB, IIB, IIIA, IVA, or VA metal or metal oxide. Embodiment 48 is the system of embodiment 47, wherein the metal or metal oxide comprises sodium (Na), magnesium (Mg), lantheum (La), Ytterbium (Y), vanadium (V), niobium (Nb), molybdenum (Mo), chromium (Cr), manganese (Mn), iron (Fe), cobalt (Co), nickel (Ni), copper (Cu), zinc (Zn), gallium (Ga), indium (In), tin (Sn), antimony (Sb), bismuth (Bi), tellurium (Te), or any combination thereof. Embodiment 49 is the system of embodiment 48, wherein the metal or metal oxide comprises Fe. Embodiment 50 is the system of any one of embodiments 46 to 49, wherein the zeolite catalyst comprises up to 10 wt. % of the metal or metal oxide, preferably 0.1 wt. % to 20 wt. %, more preferably 1 wt. % to 10 wt. %, or most preferably 3 wt. % to 7 wt. % or about 5 wt. %. Embodiment 51 is the system of any one of embodiments 27 to 50, wherein the zeolite catalyst has a Si/Al ratio of less than 100, preferably 5 to 75, more preferably 10 to 60, or most preferably 20 to 55.

The term “butane” refers to all isomers of butane unless specifically stated. Butane isomers include n-butane, isobutane (methylpropane) and mixtures thereof.

The term “catalyst” means a substance which alters the rate of a chemical reaction. “Catalytic” means having the properties of a catalyst.

The term “conversion” means the mole fraction (i.e., percent) of a reactant converted to a product or products.

The term “selectivity” refers to the percent of converted reactant that went to a specified product, for example benzene selectivity is the % of butane that converted to benzene.

The term “about” or “approximately” are defined as being close to as understood by one of ordinary skill in the art. In one non-limiting embodiment, the terms are defined to be within 10%, preferably within 5%, more preferably within 1%, and most preferably within 0.5%.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising”, “including”, “containing”, or “having” in the claims, or the specification, may mean “one,” but it is also consistent with the meaning of “one or more”, “at least one”, and “one or more than one.”

The terms “wt. %”, “vol. %”, or “mol. %” refers to a weight, volume, or molar percentage of a component, respectively, based on the total weight, the total volume of material, or total moles, that includes the component. In a non-limiting example, 10 grams of component in 100 grams of the material is 10 wt. % of component.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The methods and systems of the present invention can “comprise,” “consist essentially of,” or “consist of” particular ingredients, components, compositions, etc. disclosed throughout the specification. With respect to the transitional phase “consisting essentially of,” in one non-limiting aspect, a basic and novel characteristic of the methods and systems of the present invention are their ability to produce aromatic hydrocarbons from an alkane.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description.

### BRIEF DESCRIPTION OF THE DRAWINGS

FIG. 1 is a diagram showing some of the petrochemicals that can be produced from BTX components.

FIG. 2 is a schematic of an embodiment of a system for producing BTX from alkanes.

FIG. 3A shows graphs of Aspen thermodynamic calculations for butane conversion in percent versus temperature of various concentrations of CO₂.

FIG. 3B shows graphs of Aspen thermodynamic calculations for aromatic yield in percent versus temperature of various concentrations of CO₂.

### DETAILED DESCRIPTION OF THE INVENTION

A discovery has been made, which provides an oxidative aromatization method and systems for producing aromatic hydrocarbons from an alkane or mixtures of alkanes. The oxidative aromatization method and systems include contacting a reactant feed that includes butane and carbon dioxide (CO₂) with a zeolite catalyst under reaction conditions sufficient to produce an aromatic hydrocarbon containing product stream. The amount CO₂ present in the reactant feed can be sufficient to dehydrogenate the alkane or mixtures of alkanes. Notably, the reactant feed includes at least 10 mole % of CO₂, preferably 10 mole % to 54 mole % of CO₂. The method and systems provide benzene, toluene, and xylenes (BTX) in improved yields and selectivities where the combined selectivity of benzene, toluene, and xylenes is at least 20% at a reaction temperature of 200° C. or at least 50% at a reaction temperature of 325° C. to 600° C., at 54 mole % of CO₂, and the conversion of butane is at least 15% at a reaction temperature of 200° C. or at least 80% at a reaction temperature of 325° C. to 375° C., at 10 mole % to 54 mole % of CO₂.

In one embodiment a new route for the production of BTX from an alkane, specifically, butane in the presence of CO₂ is presented. The aromatization reaction mechanism is generally accepted to involve dehydrogenation, oligomerization, cyclization, and aromatization to aromatic hydrocarbons as illustrated in the following general reaction scheme:

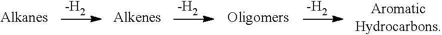

Under thermal conditions the rate limiting step is the dehydrogenation step. The direct dehydrogenation of alkanes to aromatic hydrocarbons usually occurs above 600° C., where cracking of the hydrocarbons into smaller molecules can decrease aromatic hydrocarbon selectivity and catalyst activity. Oxidative dehydrogenation (ODH) of alkanes to alkenes by oxygen has been proposed as an alternative to the high-temperature operation of dehydrogenation processes. However, the reaction is highly exothermic and heat must be removed to avoid the over-oxidation to carbon oxides. Without wishing to be bound by theory, it is believed that replacing oxygen in the reaction with CO₂ as a soft oxidant will suppress over-oxidation and the use of CO₂ can inhibit side reactions and removes coking from the catalyst surface, which further improves the yield of benzene and other aromatic hydrocarbons and accelerates the rate of dehydrogenation. Notably, the reaction can be performed at lower temperatures. In the reaction scheme above, the CO₂ promotes the formation of alkenes from the alkanes, and the zeolite catalyst promotes the formation of oligomers from the alkenes. Dehydrogenation of the oligomers to aromatic hydrocarbons can be promoted through the zeolite catalyst or through the catalytic metal loaded on the zeolite catalyst. Due to the presence of carbon dioxide, coking of the zeolite catalyst and/or sintering of catalytic metal is inhibited, thereby promoting higher selectivity and yield of aromatic products instead of formation of side products (e.g., cracking products).

These and other non-limiting aspects of the present invention are discussed in further detail in the following sections.

### A. Zeolite Catalyst

The catalysts of the present invention are capable of producing aromatic hydrocarbons from an alkane or mixtures of alkanes and carbon dioxide (CO₂) in a single pass. The catalysts used in the present invention are typically metal loaded zeolites, or hollow metal loaded zeolites. Zeolites can be an effective substrate, commercially available, and are well known in the art. Zeolites are typically microporous, aluminosilicate crystalline minerals commonly used as commercial adsorbents and catalysts in the petrochemical industry, for instance in fluid catalytic cracking and hydrocracking. The zeolite catalysts of the current invention can be a MFI, FAU, ITH BEA, MOR, LTA, MWW, CHA, MRE, MFE, or a VFI zeolite catalyst. In a specific embodiment the catalyst is a MFI zeolite catalyst, such as ZSM-5 or H-ZSM-5 which is ZSM-5 in its protonated form. The zeolite catalyst can be further loaded with a promotor element such as a catalytic metal or metal oxide and loading can be accomplished by know processes in the art, including ion exchange, impregnation, etc. Typically, zeolite catalysts used to prepare petrochemicals suffer from coke formation that leads to rapid catalyst deactivation. Encapsulating metal particles inside the hollow structure of the zeolite framework shields these metal particles from sintering which can decrease or prevent catalyst deactivation. One or more of the catalysts used in the current embodiments can include a zeolite catalyst that contains metals (e.g., metals in reduced form), metal compounds (e.g., metal oxides) or mixtures thereof (“collectively metals”) of Column 1 or 2 metals, transition metals, post-transition metals, and lanthanides (atomic number 57-71) of the Periodic Table. Non-limiting examples of transition metals and post-transition metals include chromium (Cr), molybdenum (Mo), tungsten (W), manganese (Mn), iron (Fe), ruthenium (Ru), cobalt (Co), rhodium (Rh), nickel (Ni), palladium (Pd), copper (Cu), silver (Ag), zinc (Zn), cadmium (Cd), and gallium (Ga). Specifically, the catalytic metal or metal oxide can be a group IA, IIA, VIB, VIIB, VIII, VIIIB, IB, IIB, IIIA, IVA, or VA metal or metal oxide, for example sodium (Na), magnesium (Mg), lantheum (La), Ytterbium (Y), vanadium (V), niobium (Nb), molybdenum (Mo), chromium (Cr), manganese (Mn), iron (Fe), cobalt (Co), nickel (Ni), copper (Cu), zinc (Zn), gallium (Ga), indium (In), tin (Sn), antimony (Sb), bismuth (Bi), tellurium (Te), or mixtures thereof. In a particular aspect the metal or metal oxide comprises Fe. The zeolite catalyst of the present invention can include up to 20 wt. % of the metal and/or metal oxide, from 0.1 wt. % to 20 wt. %, from 1 wt. % to 10 wt. %, or from 3 wt. % to 7 wt. % and all wt. % there between including 3.1 wt. %, 3.2 wt. %, 3.3 wt. %, 3.4 wt. %, 3.5 wt. %, 3.6 wt. %, 3.7 wt. %, 3.8 wt. %, 3.9 wt. %, 4 wt. %, 4.1 wt. %, 4.2 wt. %, 4.3 wt. %, 4.4 wt. %, 4.5 wt. %, 4.6 wt. %, 4.7 wt. %, 4.8 wt. %, 4.9 wt. %, 5 wt. %, 5.1 wt. %, 5.2 wt. %, 5.3 wt. %, 5.4 wt. %, 5.5 wt. %, 5.6 wt. %, 5.7 wt. %, 5.8 wt. %, 5.9 wt. %, 6 wt. %, 6.1 wt. %, 6.2 wt. %, 6.3 wt. %, 6.4 wt. %, 6.5 wt. %, 6.6 wt. %, 6.7 wt. %, 6.8 wt. %, and 6.9 wt. %. In a specific embodiment the zeolite catalyst includes about 5 wt. % of metal and/or metal oxide. The zeolite catalyst of the present invention can have a Si/Al ratio of less than 100, from 5 to 75, from 10 to 60, or from 20 to 55 and all ratios there between including 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, and 54. The metals used to prepare the zeolite catalyst of the present invention can be provided in varying oxidation states as metallic, oxide, hydrate, or salt forms typically depending on the propensity of each metals stability and/or physical/chemical properties. The metals in the catalyst can also exist in one or more oxidation states. Preferably the metals or metal oxides used in the preparation of the metal loaded zeolite catalyst are provided in stable oxidation states as complexes with monodentate, bidentate, tridentate, or tetradendrate coordinating ligands such as for example iodide, bromide, sulfide, thiocyanate, chloride, nitrate, azide, fluoride, hydroxide, oxalate, water, isothiocyanate, acetonitrile, pyridine, ammonia, ethylenediamine, 2,2′-bipyridine, 1,10-phenanthroline, nitrite, triphenylphosphine, cyanide, carbon monoxide, or mixtures thereof. In a preferred aspect the metals are impregnated into the zeolite catalysts as aqueous solutions of metal nitrate, metal nitrate hydrates, metal nitrate trihydrates, metal nitrate hexahydrates, and metal nitrate nonahydrates for example gallium nitrate hydrate (Ga(NO₃)₃.H₂O), nickel nitrate hexahydrate (Ni(NO₃)₂.6H₂O), copper nitrate trihydrate Cu(NO₃)₂.3H₂O, iron nitrate nonahydrate (Fe(NO₃)₃.9H₂O), cobalt nitrate hexahydrate (Co(NO₃)₂.6H₂O), chromium nitrate nonahydrate (Cr(NO₃)₃.9H₂O), and zinc nitrate hexahydrate (Zn(NO₃)₂.6H₂O). A non-limiting example of a commercial source of the above mentioned metals and metal oxides is Sigma Aldrich® (U.S.A).

### B. Methods of Making Zeolite Catalysts

The catalysts of the present invention can be prepared by processes known to those having ordinary skill in the art. By way of example, the catalysts can be prepared by liquid-liquid blending, solid-solid blending, or liquid-solid blending (e.g., any of precipitation, co-precipitation, impregnation, complexation, gelation, crystallization, microemulsion, sol-gel, solvothermal, hydrothermal, sonochemical, or combinations thereof). The metal loaded zeolite catalysts of the present invention can be prepared by impregnation of any of the above mentioned metals, metal oxides, or mixtures thereof, followed by drying and further heating in the presence of a quaternary ammonium salt or amines. Non-limiting examples of quaternary ammonium salts include tetrapropylammioum hydroxide (TPAOH), tetraethylammonium hydroxide (TEAOH), tetramethylammonium hydroxide (TMAOH), hexadecyltrimethylammonium hydroxide, dibenzyldimethylammonium hydroxide, benzyltriethylammonium hydroxide, and cetyltrimethylammonium hydroxide or alkyl derivatives thereof. Non-limiting examples of amines include, diisopropylamine (DIPA), diisopropylethylamine (DIPEA), morpholine, piperidine, pyrrolidine, diethylamine (DEA), triethylamine (TEA), or alkyl derivatives thereof. Heating the metal loaded zeolite in the presence of a quaternary ammonium salts and/or amines can have profound effects on the resultant crystal morphology (size, shape, dispersion, surface area, distribution), and thus the activity of the zeolite catalyst can be controlled. The pH of the solution can be adjusted to assist in the dissociation of the counter ion (e.g., nitrate, oxalate, chloride, sulfide etc.) from the metal oxide. Without wishing to be bound by theory, it is believed that the heating the metal loaded zeolite in the presence of the hydroxide salt can preferentially dissolve the silica in the zeolite structure. The treated zeolite and/or hollow zeolite can have an increased surface area, increased micropore surface area, an increased surface Si/Al ratio, and an increased amount of strong acid sites, all of which facilitating hydrogen transfer reactions. The quaternary ammonium salts and/or amines can be mixed or suspended with the metal impregnated zeolite catalyst from 2 ml/g zeolite to 6 ml/g zeolite, or from 3 ml/g zeolite to 5 ml/g zeolite and all value there between including about 3.1 ml/g zeolite, about 3.2 ml/g zeolite, about 3.3 ml/g zeolite, about 3.4 ml/g zeolite, about 3.5 ml/g zeolite, about 3.6 ml/g zeolite, about 3.7 ml/g zeolite, about 3.8 ml/g zeolite, about 3.9 ml/g zeolite, about 4 ml/g zeolite, about 4.1 ml/g zeolite, about 4.2 ml/g zeolite, about 4.3 ml/g zeolite, about 4.4 ml/g zeolite, about 4.5 ml/g zeolite, about 4.6 ml/g zeolite, about 4.7 ml/g zeolite, about 4.8 ml/g zeolite, about 4.9 ml/g zeolite, and in a specific embodiment 4.15 ml/g zeolite.

In a non-limiting example of the present invention, catalyst of the present invention can be prepared in a stepwise fashion by first loading the zeolite (ZSM-% with Si/Al ratio of 23, 30, or 50) with an appropriate aqueous metal solution (e.g., Ga, Ni, Cu, Fe, Co, Cr, or Zn) and then dried. In a preferred embodiment ZSM-5 is used, which can have a Si/Al ratio of 10 to 100, or 23, 30, 50, 80. In the second step, the metal loaded zeolite catalyst can be heated in the presence of an aqueous solution of a quaternary ammonium salt (e.g., TPAOH). The precipitate from either step can be collected by standard techniques, such as decanting, filtration, or centrifuging. In a preferred aspect the precipitate formed from the metal loading (e.g., impregnation) is dried at 40° C. to 60° C., specifically 50° C. under air from 8 hours to 12 hours. In another preferred aspect the templated metal loaded zeolite precipitate is formed under hydrostatic conditions (e.g., an autoclave) at a temperature from about 140° C. to about 200° C., preferably 170° C. for a period time ranging from about 12 hours to about 26 hours, preferably 24 hour. The mixture can then be centrifuged at a range from about 3000 rpm to about 7000 rpm, from about 4000 rpm to about 6000 rpm, and preferably about 5000 rpm for anywhere between 10 minutes and 30 minutes, preferably 15 minutes followed by drying overnight at temperature from about 100° C. to about 120° C., preferably 110° C. overnight to obtain a zeolite catalyst precursor. Multiple centrifugations can be performed, washing with water in between. In some aspects, the final zeolite catalysts of the present invention are prepared under oxidative conditions (i.e. calcination) and the metals included in the zeolite catalyst are present in higher oxidation states, for example as oxides. The dried templated catalyst precursor can be calcined for 2 to 24 hours, specifically about 12 hours at a temperature of 250 to 800° C., specifically about 525° C., or about 540° C. under airflow to obtain an active zeolite catalyst. The zeolite catalyst or metal loaded zeolite catalyst is not sulfided prior to use.

The zeolite catalysts of the present invention can be ground into a fine powder, micronized or nanonized to desired mesh particle size distributions, or pressed into pellets, crushed, and sieved to particle size ranges from about 100 μm to about 600 μm, from about 200 μm to about 500 μm, and preferably between 250 μm and 425 μm. Without wishing to be bound by theory, it is believed that the catalyst activity depends on the particle size of the metals in the metal loaded zeolite catalyst, which depends mainly on electronic effects, as the electron density at the active sites (on the surface) can vary due to particle size. This effect can be closely related to particle shape and the number of low coordination sites (edges and corners) on the surface as well as the composition of the catalyst.

### C. CO₂/Alkane Feed Stream

The alkane and carbon dioxide used in the present invention can be obtained from various sources. In some instances the alkane is butane including n-butane and/or isobutane where the butane and carbon dioxide may be purchased in various grades from commercial sources. In one non-limiting instance, the carbon dioxide can be obtained from a waste or recycle gas stream (e.g. from a plant on the same site, like for example from ammonia synthesis) or after recovering the carbon dioxide from a gas stream. A benefit of recycling such carbon dioxide as a starting material in the process of the invention is that it can reduce the amount of carbon dioxide emitted to the atmosphere (e.g., from a chemical production site). In a first aspect the mole % ratio of CO₂ to the alkane or CO₂ to the mixture of alkanes is 0.1 to 10, preferably 0.1 to 7, more preferably 0.1 to 5, or most preferably 0.1 to 3 and all ratios in between including 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, and 2.9 mole % ratio of CO₂ to the alkane or CO₂ to the mixture of alkanes. In a second aspect the reactant feed can include at least 10 mole % of CO₂, preferably 10 mole % to 54 mole % of CO₂ and all mole % in between including 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, and 53 mole % of CO₂. In another aspect of the method the reactant feed comprises substantially stoichiometric amounts of CO₂. The alkane used in the method can be butane or the mixture of alkanes includes at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 mole %, or more, including 91, 92, 93, 94, 95, 96, 97, 98, or 99 mole % of butane. The butane is preferably n-butane. The remainder of the reactant gas stream can include another gas or gases provided the gas or gases are inert, such as helium (He), argon (Ar), or nitrogen (N₂), and do not negatively affect the reaction. All possible percentages of alkane±CO₂±inert gas are anticipated in the current embodiments as having the described CO₂/alkane ratios herein. In another embodiment when the alkane is butane, the butane can be a mixture of n-butane and isobutane and/or the reactant feed does not include any other alkane other than butane or a mixture of n-butane and isobutane. For example, in one instance the reactant feed stream includes 13 vol. % CO₂, 11.5 vol. % butane, and 74.7 vol. % N₂. In another instance the reactant feed stream includes 54.5 vol. % CO₂ and 45.5 vol. % butane. Preferably, the reactant mixture is highly pure and substantially devoid of impurities.

### D. Aromatic Hydrocarbon Production System

Conditions sufficient for the aromatization reaction of an alkane or mixture of alkanes include temperature, time, space velocity, and pressure. The temperature range for the aromatization reaction is a least 200° C. and ranges from 200° C. to 700° C., from 200° C. to 600° C., from 250° C. to 550° C., and in a specific embodiment from 275° C. to 540° C. and all temperatures in between including 276° C., 277° C., 278° C., 279° C., 280° C., 281° C., 282° C., 283° C., 284° C., 285° C., 286° C., 287° C., 288° C., 289° C., 290° C., 291° C., 292° C., 293° C., 294° C., 295° C., 296° C., 297° C., 298° C., 300° C., 331° C., 302° C., 303° C., 304° C., 305° C., 306° C., 307° C., 308° C., 309° C., 310° C., 311° C., 312° C., 313° C., 314° C., 315° C., 316° C., 317° C., 318° C., 319° C., 320° C., 321° C., 322° C., 323° C., 324° C., 325° C., 326° C., 327° C., 328° C., 329° C., 330° C., 331° C., 332° C., 333° C., 334° C., 335° C., 336° C., 337° C., 338° C., 339° C., 340° C., 341° C., 342° C., 343° C., 344° C., 345° C., 346° C., 347° C., 348° C., 349° C., 350° C., 351° C., 352° C., 353° C., 354° C., 355° C., 356° C., 357° C., 358° C., 359° C., 360° C., 361° C., 362° C., 363° C., 364° C., 365° C., 366° C., 367° C., 368° C., 369° C., 370° C., 371° C., 372° C., 373° C., 374° C., 375° C., 376° C., 377° C., 378° C., 379° C., 380° C., 381° C., 382° C., 383° C., 384° C., 385° C., 386° C., 387° C., 388° C., 389° C., 390° C., 391° C., 392° C., 393° C., 394° C., 395° C., 396° C., 397° C., 398° C., 399° C., 400° C., 401° C., 402° C., 403° C., 404° C., 405° C., 406° C., 407° C., 408° C., 409° C., 410° C., 411° C., 412° C., 413° C., 414° C., 415° C., 416° C., 417° C., 418° C., 419° C., 420° C., 421° C., 422° C., 423° C., 424° C., 425° C., 426° C., 427° C., 428° C., 429° C., 430° C., 431° C., 432° C., 433° C., 434° C., 435° C., 436° C., 437° C., 438° C., 439° C., 440° C., 441° C., 442° C., 443° C., 444° C., 445° C., 446° C., 447° C., 448° C., 449° C., 450° C., 451° C., 452° C., 453° C., 454° C., 455° C., 456° C., 457° C., 458° C., 459° C., 460° C., 461° C., 462° C., 463° C., 464° C., 465° C., 466° C., 467° C., 468° C., 469° C., 470° C., 471° C., 472° C., 473° C., 474° C., 475° C., 476° C., 477° C., 478° C., 479° C., 480° C., 481° C., 482° C., 483° C., 484° C., 485° C., 486° C., 487° C., 488° C., 489° C., 490° C., 491° C., 492° C., 493° C., 494° C., 495° C., 496° C., 497° C., 498° C., 499° C., 500° C., 501° C., 502° C., 503° C., 504° C., 505° C., 506° C., 507° C., 508° C., 509° C., 510° C., 511° C., 512° C., 513° C., 514° C., 515° C., 516° C., 517° C., 518° C., 519° C., 520° C., 521° C., 522° C., 523° C., 524° C., 525° C., 526° C., 527° C., 528° C., 529° C., 530° C., 531° C., 532° C., 533° C., 534° C., 535° C., 536° C., 537° C., 538° C., 539° C. The gas hourly space velocity (GHSV) for the aromatization reaction can range from about 500 h⁻¹ to 10,000 h⁻¹ and in a specific embodiment from about 500 h⁻¹ to 5000 h⁻¹ and all GHSV in between including 600 h⁻¹, 700 h⁻¹, 800 h⁻¹, 900 h⁻¹, 1000 h⁻¹, 1100 h⁻¹, 1200 h⁻¹, 1300 h⁻¹, 1400 h⁻¹, 1500 h⁻¹, 1600 h⁻¹, 1700 h⁻¹, 1800 h⁻¹, 1900 h⁻¹, 2000 h⁻¹, 2100 h⁻¹, 2200 h⁻¹, 2300 h⁻¹, 2400 h⁻¹, 2500 h⁻¹, 2600 h⁻¹, 2700 h⁻¹, 2800 h⁻¹, 2900 h⁻¹, 3000 h⁻¹, 3100 h⁻¹, 3200 h⁻¹, 3300 h⁻¹, 3400 h⁻¹, 3500 h⁻¹, 3600 h⁻¹, 3700 h⁻¹, 3800 h⁻¹, 3900 h⁻¹, 4000 h⁻¹, 4100 h⁻¹, 4200 h⁻¹, 4300 h⁻¹, 4400 h⁻¹, 4500 h⁻¹, 4600 h⁻¹, 4700 h⁻¹, 4800 h⁻¹, and 4900 h⁻¹. The average pressure for the aromatization reaction can range from about 0.5 bar to about 5 bar, and in a specific embodiment from about 1 bar to about 3 bar and all pressures there between including 1.1 bar, 1.2 bar, 1.3 bar, 1.4 bar, 1.5 bar, 1.6 bar, 1.7 bar, 1.8 bar, 1.9 bar, 2 bar, 2.1 bar, 2.2 bar, 2.3 bar, 2.4 bar, 2.5 bar, 2.6 bar, 2.7 bar, 2.8 bar, and 2.9 bar, or more. The upper limit on pressure can be determined by the reactor used. The conditions for the aromatization reaction can be varied based on the type of the reactor.

In another aspect, the reaction can be carried out over the zeolite catalyst of the current invention having the particular aromatic hydrocarbon selectivity and butane conversion. In one embodiment the aromatic hydrocarbon containing product stream can include one or more of benzene, toluene, and xylenes (BTX), where the combined selectivity of one or more of benzene, toluene, and xylenes is at least 20% including 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, and 99% at a reaction temperature of 200° C. or at least 50% including 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, and 99% at a reaction temperature of 325° C. to 600° C. and all temperatures in between including 326° C., 327° C., 328° C., 329° C., 330° C., 331° C., 332° C., 333° C., 334° C., 335° C., 336° C., 337° C., 338° C., 339° C., 340° C., 341° C., 342° C., 343° C., 344° C., 345° C., 346° C., 347° C., 348° C., 349° C., 350° C., 351° C., 352° C., 353° C., 354° C., 355° C., 356° C., 357° C., 358° C., 359° C., 360° C., 361° C., 362° C., 363° C., 364° C., 365° C., 366° C., 367° C., 368° C., 369° C., 370° C., 371° C., 372° C., 373° C., 374° C. 375° C., 376° C., 377° C., 378° C., 379° C., 380° C., 381° C., 382° C., 383° C., 384° C., 385° C., 386° C., 387° C., 388° C., 389° C., 390° C., 391° C., 392° C., 393° C., 394° C., 395° C., 396° C., 397° C., 398° C., 399° C., 400° C., 401° C., 402° C., 403° C., 404° C., 405° C., 406° C., 407° C., 408° C., 409° C., 410° C., 411° C., 412° C., 413° C., 414° C., 415° C., 416° C., 417° C., 418° C., 419° C., 420° C., 421° C., 422° C., 423° C., 424° C., 425° C., 426° C., 427° C., 428° C., 429° C., 430° C., 431° C., 432° C., 433° C., 434° C., 435° C., 436° C., 437° C., 438° C., 439° C., 440° C., 441° C., 442° C., 443° C., 444° C., 445° C., 446° C., 447° C., 448° C., 449° C., 450° C., 451° C., 452° C., 453° C., 454° C., 455° C., 456° C., 457° C., 458° C., 459° C., 460° C., 461° C., 462° C., 463° C., 464° C., 465° C., 466° C., 467° C., 468° C., 469° C., 470° C., 471° C., 472° C., 473° C., 474° C., 475° C., 476° C., 477° C., 478° C., 479° C., 480° C., 481° C., 482° C., 483° C., 484° C., 485° C., 486° C., 487° C., 488° C., 489° C., 490° C., 491° C., 492° C., 493° C., 494° C., 495° C., 496° C., 497° C., 498° C., 499° C., 500° C., 501° C., 502° C., 503° C., 504° C., 505° C., 506° C., 507° C., 508° C., 509° C., 510° C., 511° C., 512° C., 513° C., 514° C., 515° C., 516° C., 517° C., 518° C., 519° C., 520° C., 521° C., 522° C., 523° C., 524° C., 525° C., 526° C., 527° C., 528° C., 529° C., 530° C., 531° C., 532° C., 533° C., 534° C., 535° C., 536° C., 537° C., 538° C., 539° C., 540° C., 541° C., 542° C., 543° C., 544° C., 545° C., 546° C., 547° C., 548° C., 549° C., 550° C., 551° C., 552° C., 553° C., 554° C., 555° C., 556° C., 557° C., 558° C., 559° C., 560° C., 561° C., 562° C., 563° C., 564° C., 565° C., 566° C., 567° C., 568° C., 569° C., 570° C., 571° C., 572° C., 573° C., 574° C., 575° C., 576° C., 577° C., 578° C., 579° C., 580° C., 581° C., 582° C., 583° C., 584° C., 585° C., 586° C., 587° C., 588° C., 489° C., 590° C., 591° C., 592° C., 593° C., 594° C., 595° C., 596° C., 597° C., 598° C., 599° C., at 54 mole % of CO₂. The conversion of butane is at least 15% including 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, and 99% at a reaction temperature of 200° C. or at least 50% including 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, and 99% at a reaction temperature of 200° C. or at least 80% including 81, 82, 83, 84, 85, 86, 87, 88, 89, 90, 91, 92, 93, 94, 95, 96, 97, 98, and 99% at a reaction temperature of 325° C. to 375° C. and all temperature in between including 326° C., 327° C., 328° C., 329° C., 330° C., 331° C., 332° C., 333° C., 334° C., 335° C., 336° C., 337° C., 338° C., 339° C., 340° C., 341° C., 342° C., 343° C., 344° C., 345° C., 346° C., 347° C., 348° C., 349° C., 350° C., 351° C., 352° C., 353° C., 354° C., 355° C., 356° C., 357° C., 358° C., 359° C., 360° C., 361° C., 362° C., 363° C., 364° C., 365° C., 366° C., 367° C., 368° C., 369° C., 370° C., 371° C., 372° C., 373° C., and 374° C. at 10 mole % to 54 mole % of CO₂ and all mole % in between including 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, and 53 mole % of CO₂. Xylenes exists as several dimethylbenzene constitutional isomers including ortho-xylene (1,2-dimethylbenzene), meta-xylene (1,3-dimethylbenzene), and para-xylene (1,4-dimethylbenzene). Other alkylbenzenes formed during the aromatization reaction include one or more of ethylbenzene (EB), trimethylbenzenes (TMB), isopropylbenzene, n-propylbenzene, 1-methyl-3-ethylbenzene, 1-methyl-4-ethylbenzene, 1,2,3-trimethylbenzene (hemellitene), 1,2,4-trimethylbenzene (pseudocumene), and 1,3,5-trimethylbenzene (mesitylene), tert-butylbenzene, isobutylbenzene, sec-butylbenzene, n-butylbenzene, 1-methyl-2-ethylbenzene, 1-methyl-3-ethylbenzene, 1-methyl-4-isopropylbenzene, 1-methyl-2-n-propylbenzene, 1-methyl-4-n-propylbenzene, 1-methyl-3-n-propylbenzene, 1,3-dimethyl-5-ethylbenzene, 1,4-dimethyl-2-ethylbenzene 1,2-dimethyl-4-ethylbenzene, 1,3-dimethyl-2-ethylbenzene, 1,2,4,5,-tetramethylbenzene, 1,2-diethylbenzene, 2-methylbutylbenzene, 1,2-diethylbenzene, 2-methylbutylbenzene, tert-1-butyl-2-methylbenzene, tert-1-butyl-4-ethylbenzene, 1,2,4-triethylbenzene, 1,3,5-triethylbenzene, n-hexylbenzene. A mixture of hemellitene, pseudocumene, and mesitylene can be referred to as TMB. The composition of benzene, toluene, ethylbenzene, and xylenes is referred to as BTEX.

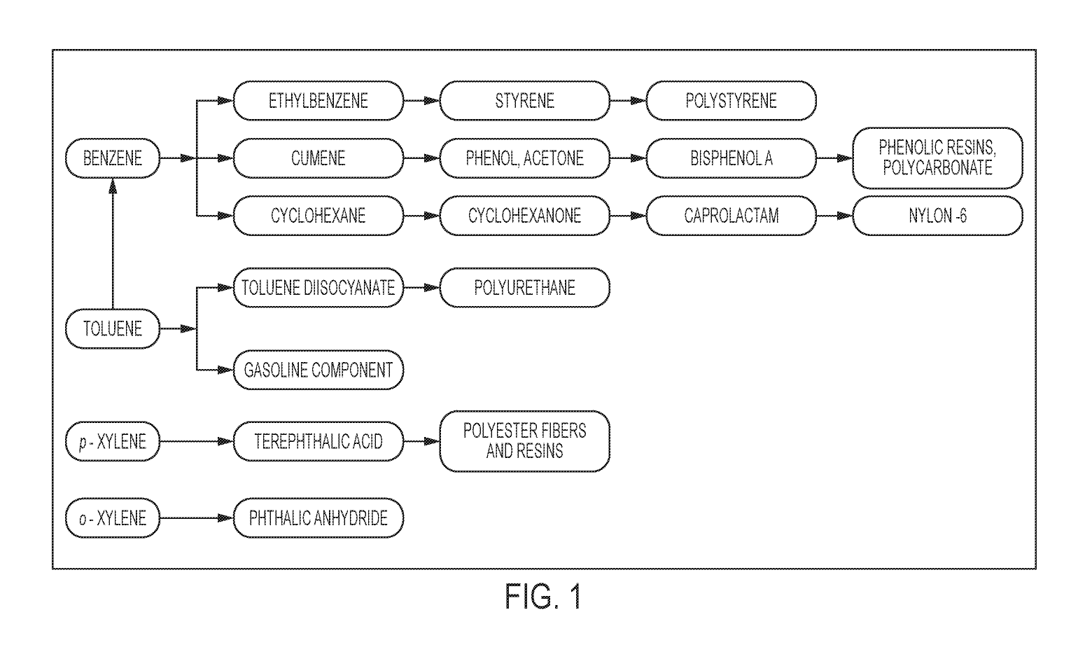

The zeolite catalyst may be used for prolonged periods of time without changing or re-supplying new catalyst or preforming catalyst regeneration. This is due to the stability or slower deactivation of the catalysts of the present invention. Therefore, in one aspect the reaction can be performed wherein one pass aromatic selectivity is 1 to 10%, preferably, 10 to 90%, or more preferably from 20 to 60% after 3 hours to 10 hours on the stream. In another aspect the one pass butane conversion is 10 to 100% after 3 hours to 10 hours on the stream and the catalysts of the present invention remain 50 to 90% active, preferably 60 to 80% active, after 10 hours of time on the stream. The method can further include collecting or storing the produced aromatic hydrocarbons along with using the produced aromatic hydrocarbons, after separation, as a feed source for petrochemical products or a polymer. By way of example only, FIG. 1 provides non-limiting uses of the constituents of BTX produced from the method and systems of the present invention. In some aspects before use, the zeolite catalysts of the present invention are treated under oxidative conditions (i.e. calcination in air) and the metals included in the zeolite catalyst are present in higher oxidation states, for example as oxides. The zeolite catalyst can be calcined for 2 to 24 hours, specifically about 12 hours at a temperature of 250 to 800° C., specifically about 525° C. under airflow to obtain the desired catalyst oxidation state.

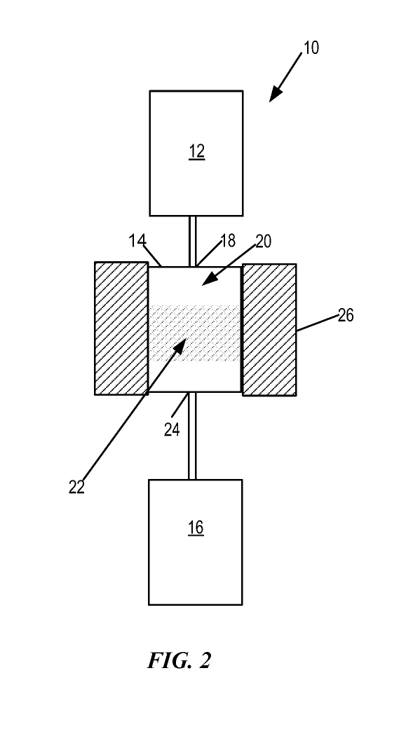

Referring to FIG. 2, a system 10 is illustrated, which can be used to convert an alkane or mixture of alkanes and carbon dioxide to aromatic hydrocarbons using the zeolite catalysts of the present invention. The system 10 can include a feed source 12, a reactor 14, and a collection device 16. The feed source 12 can be configured to be in fluid or gas communication with the reactor 14 via an inlet 18 on the reactor. The feed can include any of the reactants disclosed throughout the current disclosure including, but not limited to, mixtures of an alkane or mixture of alkanes and a gas such as butane and carbon dioxide. As explained above, the feed source 12 can be configured such that it regulates the amount of reactant feed entering the reactor 14. As shown, the alkane and carbon dioxide feed source 12 is one unit feeding into one inlet 18, however, it should be understood that the number of inlets and/or separate feed sources can be adjusted to reactor sizes and/or configurations. The reactor 14 can include a reaction zone 20 having the zeolite catalyst 22 of the present invention. The reactor can include various automated and/or manual controllers, valves, heat exchangers, gauges, etc. necessary for the operation of the reactor. The reactor can be have the necessary insulation and/or heat exchangers to heat or cool the reactor as necessary. The amounts of the alkane and carbon dioxide feed and the zeolite catalyst 22 used can be modified as desired to achieve a given amount of product produced by the system 10. Non-limiting examples of continuous flow reactors that can be used include fixed-bed reactors, fluidized reactors, bubbling bed reactors, slurry reactors, rotating kiln reactors, moving bed reactors or any combinations thereof when two or more reactors are used.

In preferred aspects, reactor 14 is a continuous flow fixed-bed reactor. The reactor 14 can include an outlet 24 configured to be in fluid communication with the reaction zone and configured to remove a first hydrocarbon product stream comprising aromatic hydrocarbons from the reaction zone 20. Reaction zone 20 can further include the reactant feed and the first product stream. The products produced can include benzene, toluene, and xylene (BTX). In some aspects, the catalyst can be included in the product stream. The collection device 16 can be in fluid communication with the reactor 14 via the outlet 24. Both the inlet 18 and the outlet 24 can be opened and closed as desired. The collection device 16 can be configured to store, further process, or transfer desired reaction products (e.g., benzene, toluene, and xylenes) for other uses. In a non-limiting example, collection device can be a separation unit or a series of separation units that are capable of separating the liquid components from the gaseous components from the product stream. The resulting aromatic hydrocarbons can be sold, stored or used in other processing units as a feed source. Still further, the system 10 can also include a heating/cooling source 26. The heating/cooling source 26 can be configured to heat or cool the reaction zone 20 to a temperature sufficient (e.g., 400° C. or 600° C.) to convert an alkane in the reactant feed to aromatic hydrocarbons. Non-limiting examples of a heating/cooling source 20 can be a temperature controlled furnace or an external, electrical heating block, heating coils, or a heat exchanger.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Example 1

### Theoretical Calculations

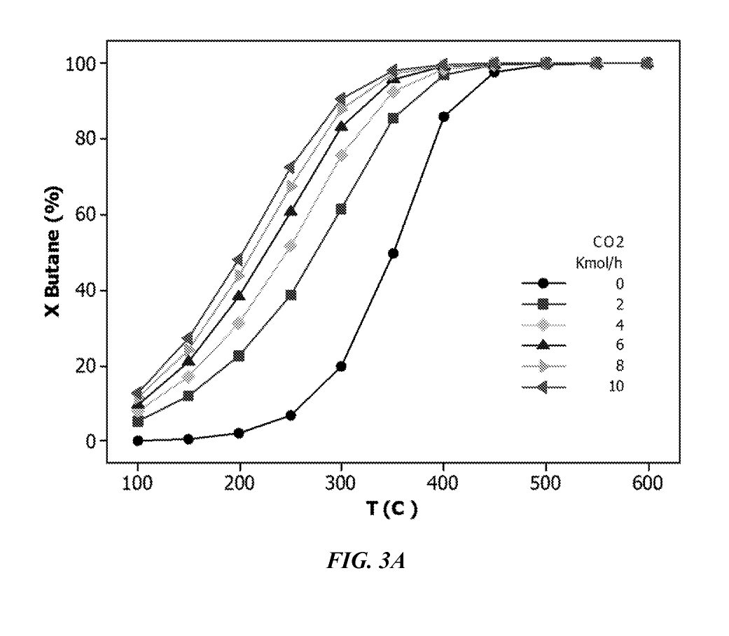

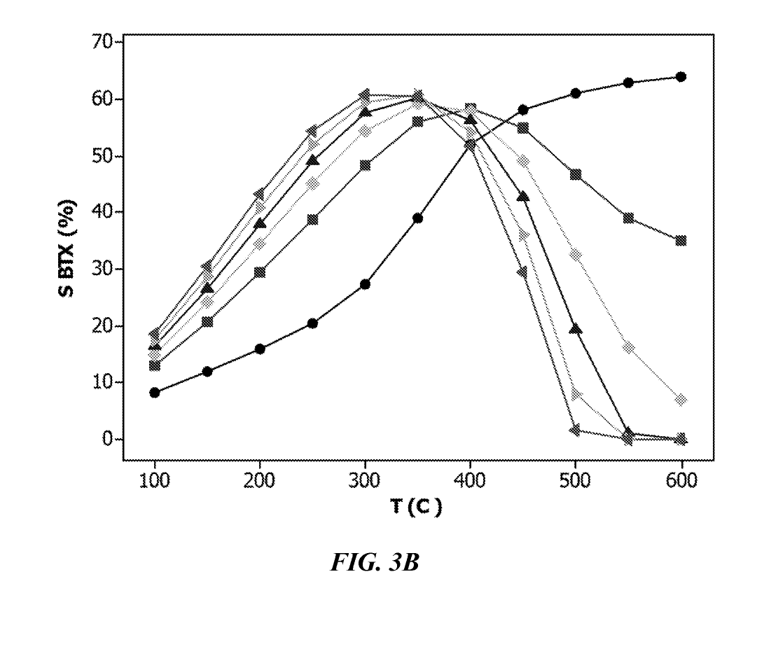

Aspen (Aspen Plus V8.0, AspenTech, Burlington, Mass., USA) thermodynamic calculations were performed to assess the addition of CO₂ to n-butane. FIG. 3A shows plots of butane conversion (X_{C4}) in percentages versus temperature at a CO₂ flow of 0 to 10 kmol/h. FIG. 3B shows plots of BTX (S_{BTX}) selectivity in percentage versus temperature. In FIGS. 3A and 3B, the circle monikers represent data for 0 kmol/h, the square monikers represent data for 2 kmol/h, the diamond monikers represent data for 4 kmol/h, the triangle monikers represent data for 6 kmol/h, the right pointing triangle moniker represent 8 kmol/h, and the left pointing triangle monikers represent data for 10 kmol/hr. As depicted in FIGS. 3A and 3B, the Aspen thermodynamic calculation showed that the addition of CO₂ to n-butane had a positive effects on the BTX production (increasing the CO₂ amount in the feed increased the BTX formation rate) at lower temperatures. Also, the calculations showed that an increase in butane conversion and BTX selectivity at 350° C. are enhanced with CO₂ in the feed as shown in Table 1.

TABLE 1

| | X_{C4} | S_{BTXe} |
|---|---|---|
| C₄H₁₀/CO₂ = 1/0 | 50 | 40 |
| C₄H₁₀/CO₂ = 1/4 | 93 | 60 |

### Catalyst Preparation

### Example 2

### General Synthesis of Hollow Metal/ZSM-5

Zeolites (ZSM-% with Si/Al ratio of 23, 30 and 50) were used as a catalyst support. The support was impregnated with aqueous solutions of Ga, Ni, Cu, Fe, Co, Cr, Zn (Ga(NO₃)₃.H₂O, Ni(NO₃)₂.6H₂O, Cu(NO₃)₂.3H₂O, Fe(NO₃)₃.9H₂O, Co(NO₃)₂.6H₂O, Cr(NO₃)₃.9H₂O, Zn(NO₃)₂.6H₂O). The suspension was dried at 50° C. under air overnight. The impregnated zeolite was then suspended with tetrapropylammonium hydroxide (4.15 ml/g zeolite) and water (3.33 ml/g zeolite). The mixture was transferred into Teflon-lined autoclave and heated at 170° C. under static conditions for 24 h. The solid was recovery by centrifugation and washed with water, this operation was repeated 3 times. The resulting solid was dried overnight at 110° C. and then calcined for 12 h at 525° C. in air.

### Example 3

### Synthesis of Hollow Ga/ZSM-5

ZSM-5 was impregnated with an aqueous solution of Ga(NO₃)₃.H₂O to produce a catalyst have 3 wt. % Ga based on the total weight of the catalyst. The suspension was dried at 50° C. under air overnight. The impregnated zeolite was then suspended with tetrapropylammonium hydroxide (4.15 ml/g zeolite) and water (3.33 ml/g zeolite). The mixture was transferred into Teflon-lined autoclave and heated at 170° C. under static conditions for 24 h. The solid was recovery by centrifugation and washed with water, this operation was repeated 3 times. The resulting solid was dried overnight at 110° C. and then calcined for 12 h at 525° C. in air to yield a Ga/ZSM-5 catalyst.

### Example 4

### Synthesis of Hollow Zn/ZSM-5

ZSM-5 was impregnated with an aqueous solution of Zn(NO₃)₃.6H₂O to produce a catalyst have 3 wt. % Zn based on the total weight of the catalyst. The suspension was dried at 50° C. under air overnight. The impregnated zeolite was then suspended with tetrapropylammonium hydroxide (4.15 ml/g zeolite) and water (3.33 ml/g zeolite). The mixture was transferred into Teflon-lined autoclave and heated at 170° C. under static conditions for 24 h. The solid was recovery by centrifugation and washed with water, this operation was repeated 3 times. The resulting solid was dried overnight at 110° C. and then calcined for 12 h at 525° C. in air to yield a Zn/ZSM-5 catalyst.

### Example 5

### Synthesis of Hollow Ga—Zn/ZSM-5

ZSM-5 was impregnated with an aqueous solution of Zn(NO₃)₃.H₂O and Zn(NO₃)₃.6H₂O to produce a catalyst have 3 wt. % Ga and 3 wt. % Zn based on the total weight of the catalyst. The suspension was dried at 50° C. under air overnight. The impregnated zeolite was then suspended with tetrapropylammonium hydroxide (4.15 ml/g zeolite) and water (3.33 ml/g zeolite). The mixture was transferred into Teflon-lined autoclave and heated at 170° C. under static conditions for 24 h. The solid was recovery by centrifugation and washed with water, this operation was repeated 3 times. The resulting solid was dried overnight at 110° C. and then calcined for 12 h at 525° C. in air to yield a Ga—Zn/ZSM-5 catalyst.

### Example 5

### Synthesis of Hollow Fe/ZSM-5

ZSM-5 was impregnated with an aqueous solution of Fe(NO₃)₃.6H₂O to produce a Fe/ZSM-5 based on the total weight of the catalyst. The suspension was dried at 50° C. under air overnight. The impregnated zeolite was then suspended with tetrapropylammonium hydroxide (4.15 ml/g zeolite) and water (3.33 ml/g zeolite). The mixture was transferred into Teflon-lined autoclave and heated at 170° C. under static conditions for 24 h. The solid was recovery by centrifugation and washed with water, this operation was repeated 3 times. The resulting solid was dried overnight at 110° C. and then calcined for 12 h at 525° C. in air to yield a Fe/ZSM-5 catalyst.

### Example 7

### Catalyst Testing General Procedure

Catalyst testing was performed in a fixed bed reactor with 0.5 cm in diameter and 25 cm in length. The effluent of the reactor is connected to Agilent gas chromatography (GC) 7890 A for online gas analysis. Catalyst was pressed into pellets then crushed and sieved between 250-425 μm. A catalyst sieved fraction (0.25 ml) was placed on top of porous plate inside the reactor. Prior to the reaction test, the catalyst was calcined at 550° C. for 3 h in air. A mixture of butane or a mixture of butane and inert gas (He or CO₂) with weight hourly space velocity (WHSV) of 1000 h⁻¹ was introduced into the reactor at atmospheric pressure and 540° C. The Fe/ZSM-5 catalyst was tested at 450° C. and a WHSV of 3000 h⁻¹. Butane conversion as well as products selectivity and yield were calculated as follows:

$${X}_{Isopro} = \frac{{Σ}_{1}^{i} {nC}_{i}}{{[ butane ]}_{in}}$$

$${S}_{Ci} = \frac{[ {C}_{i} ]}{{Σ}_{1}^{i} {nC}_{i}}$$

$${Y}_{Ci} = {X}_{butane} \times {S}_{Ci}$$

where n=number of carbon atoms.

Table 2 lists the benzene yield for n-butane aromatization over catalysts from Examples 3-5 in the presence and absence of CO₂. From the data in Table 2, it was determined that when CO₂ was used as a co-reactant, the yield percent of benzene increased. Notably, a zinc-containing catalyst demonstrated the highest increase in yield.

TABLE 2

Benzene Yield (%)

| Catalyst | He | CO₂ |
|---|---|---|
| 3%Ga/ZSM-5 | 13 | 18 |
| 3%Zn/ZSM-5 | 11 | 26 |
| 3%Ga—3%Zn/ZSM-5 | 14 | 31 |
| 3%Ga/ZSM-5 | 13 | 18 |
| Fe/ZSM-5 | 4.2 | 6.6 |

1. An oxidative aromatization method for producing aromatic hydrocarbons from alkanes, the method comprising contacting a reactant feed comprising an alkane, or a mixture of alkanes, and carbon dioxide (CO₂) with a zeolite catalyst under reaction conditions sufficient to produce an aromatic hydrocarbon containing product stream, wherein the amount of CO₂ present in the reactant feed is sufficient to dehydrogenate the alkane or mixture of alkanes, and wherein the reactant feed comprises at least 10 mole % of CO₂.

2. The method of claim 1, wherein the mole % ratio of CO₂ to the alkane or CO₂ to the mixture of alkanes is 0.1 to 10.

3. The method of claim 1, wherein the reactant feed comprises substantially stoichiometric amounts of CO₂.

4. The method of claim 1, wherein the alkane is butane or wherein the mixture of alkanes comprises at least 50, 60, 70, 80, or 90 mole %, or more, of butane.

5. The method of claim 4, wherein the aromatic hydrocarbon containing product stream comprises benzene, toluene, and xylene, and wherein the combined selectivity of benzene, toluene, and xylene is at least 20% at a reaction temperature of 200° C. or at least 50% at a reaction temperature of 325° C. to 600° C., at 54 mole % of CO₂.

6. The method of claim 5, wherein the conversion of butane is at least 15% at a reaction temperature of 200° C. or at least 80% at a reaction temperature of 325° C. to 450° C., at 10 mole % to 54 mole % of CO₂.

7. The method of claim 1, wherein the reaction conditions include a temperature of at least 200° C., and/or a pressure of 0.5 to 5 bar, or 1 to 3 bar, and a gas hourly space velocity (GHSV) of 500 h⁻¹ to 10,000 h⁻¹.

8. The method of claim 1, wherein the reactants in the reactant feed are in the gas phase.

9. The method of claim 8, wherein the reactant feed further comprises a carrier gas.

10. The method of claim 1, wherein the aromatic hydrocarbon containing product stream comprises benzene, toluene, and xylene.

11. The method of claim 1, wherein the reactant feed consists essentially of or consists of the alkane, or a mixture of alkanes, and carbon dioxide (CO₂).

12. The method of claim 1, wherein the reactant feed does not include any other alkane other than butane.

13. The method of claim 1, wherein the reactant feed does not include oxygen (O₂).

14. The method of claim 1, wherein the zeolite catalyst is a MFI, FAU, ITH BEA, MOR, LTA, MWW, CHA, MRE, MFE, or a VFI zeolite catalyst.

15. The method of claim 14, wherein the MFI zeolite catalyst is ZSM-5, H-ZSM-5, a hollow ZSM-5, or a hollow H-ZSM-5.

16. The method of claim 1, wherein the zeolite catalyst is loaded with a catalytic metal or metal oxide.

17. The method of claim 16, wherein the catalytic metal or metal oxide is a group IA, IIA, VIB, VIIB, VIII, VIIIB, IB, IIB, IIIA, IVA, or VA metal or metal oxide.

18. The method of claim 17, wherein the metal or metal oxide comprises sodium (Na), magnesium (Mg), lantheum (La), Ytterbium (Y), vanadium (V), niobium (Nb), molybdenum (Mo), chromium (Cr), manganese (Mn), iron (Fe), cobalt (Co), nickel (Ni), copper (Cu), zinc (Zn), gallium (Ga), indium (In), tin (Sn), antimony (Sb), bismuth (Bi), tellurium (Te), or any combination thereof.

19. The method of claim 18, wherein the metal or metal oxide comprises Fe, Ga, Zn, or Ga and Zn.

20. The method of claim 1, wherein the zeolite catalyst comprises up to 20 wt. % of the metal or metal oxide and/or has a Si/Al ratio of less than 100.
